(19) United States
(12) Patent Application Publication
(10) Pub. No.: US 2025/0128241 A1
(43) Pub. Date: Apr. 24, 2025

(54) PROCESS AND CATALYST FOR OXIDATIVE ESTERIFICATION WITH LONG-LIFE CATALYST

(71) Applicants: Dow Global Technologies LLC, Midland, MI (US); Rohm and Haas Company, Collegeville, PA (US)

(72) Inventors: Kirk W. Limbach, Dresher, PA (US); Christopher D. Frick, Pottstown, PA (US); Wen Sheng Lee, Midland, MI (US); Victor J. Sussman, Midland, MI (US)

(21) Appl. No.: 18/695,388

(22) PCT Filed: Oct. 5, 2022
(86) PCT No.: PCT/US2022/045723

§ 371 Date: Mar. 26, 2024

Related U.S. Application Data
(63) Continuation of application No. 63/253,556, filed on Oct. 8, 2021.

(51) Int. Cl. B01J 23/52; B01J 21/08; B01J 35/30; B01J 37/02; B01J 37/08; C07C 67/44

(52) U.S. Cl. CPC B01J 23/52; B01J 21/08; B01J 35/393; B01J 37/0201; B01J 37/0236; B01J 37/08; C07C 67/44

(57) ABSTRACT

A catalyst comprises gold particles and titanium-containing particles. The catalyst comprises gold particles that are within at least 15 nm of at least one titanium-containing particle. The gold particles have an average diameter of less than 15 nm and a standard deviation of +/−5 nm. A method for preparing methyl methacrylate from methacrolein and methanol using the catalyst is also disclosed.

### BACKGROUND OF THE INVENTION

The invention relates to a catalyst and method for preparing methyl methacrylate from methacrolein and methanol.

Heterogeneous catalysts having noble metals concentrated in an outer region of the catalyst are known, see, e.g., U.S. Pat. No. 6,228,800, for use in producing methyl methacrylate.

WO 2019/057458 discloses a process for preparing a carboxylic ester from an aldehyde via heterogeneous catalysis in a liquid phase in the presence of a catalyst particle. The catalyst particle consists of 0.1% to 3% by weight of gold, 25% to 99.8% by weight of TiO₂, 0% to 50% by weight of silicon oxide, 0% to 25% by weight of Al₂O₃, 0% to 25% by weight of at least one oxide of an alkali metal, an alkaline earther metal, a rare earth metal, and/or zirconium, 0% to 20% by weight of at least one oxide selected from the group consisting of an iron oxide, a zinc oxide, and a cobalt oxide, and 0% to 5% by weight of at least one other component. The catalyst is preferably composed predominantly or exclusively of gold and TiO₂.

However, there is a need for an improved catalyst and process for production of methyl methacrylate that is effective and active for longer lifespans.

### SUMMARY OF THE INVENTION

One aspect of the present invention relates to a catalyst comprising gold particles and titanium-containing particles, wherein the catalyst comprises gold particle that are within at least 15 nm of at least one titanium-containing particle, and wherein the gold particles have an average diameter of less than 15 nm and a standard deviation of +/−5 nm.

Another aspect of the present invention relates to a method for preparing methyl methacrylate from methacrolein and methanol; said method comprising contacting in a reactor a mixture comprising methacrolein, methanol and oxygen in the presence of a catalyst comprising gold particles and titanium-containing particles, wherein the catalyst comprises gold particles that are within at least 15 nm of at least one titanium-containing particle, and wherein the gold particles have an average diameter of less than 15 nm and a standard deviation of +/−5 nm.

### DETAILED DESCRIPTION OF THE INVENTION

All percentage compositions are weight percentages (wt %), and all temperatures are in ° C., unless otherwise indicated. Averages are arithmetic averages unless otherwise indicated. The “catalyst center” is the centroid of the catalyst particle, i.e., the mean position of all points in all coordinate directions. A diameter is any linear dimension passing through the catalyst center and the average diameter is the arithmetic mean of all possible diameters. The aspect ratio is the ratio of the longest to the shortest diameters. Unless otherwise stated, the average diameter of a particle refers to the average diameter of the particle after the catalyst has been prepared and before the catalyst has been used. An aged catalyst is a catalyst that has been used.

The catalyst of the present invention comprises gold particles and titanium-containing particles.

The gold particles and titanium-containing particles are preferably disposed on an outer surface of a support material.

Preferably, the gold particles are within at least 15 nm of a titanium-containing particle. As used herein, the phrase “within at least X nm” means that an edge of a gold particle is within X nm of an edge of the titanium-containing particle nearest the gold particle. Preferably, each gold particle is within at least 10 nm of a titanium-containing particle, more preferably within at least 8 nm of a titanium-containing particle, and even more preferably within at least 6 nm of a titanium-containing particle.

More preferably, the catalyst comprises gold particles that are within at least 15 nm of two titanium-containing particles, i.e., an edge of the gold particle is within at least 15 nm of an edge of the two titanium-containing particles nearest the gold particle. Preferably, the catalyst comprises gold particles within at least 10 nm of two titanium-containing particles, more preferably within at least 8 nm of two titanium-containing particles, and even more preferably within at least 6 nm of two titanium-containing particles.

Even more preferably, the catalyst comprises gold particles within at least 15 nm of at least three titanium-containing particles, i.e., an edge of the gold particle is within at least 15 nm of an edge of at least the three titanium-containing particles nearest the gold particle. Preferably, the catalyst comprises gold particles within at least 10 nm of at least three titanium-containing particles, more preferably within at least 8 nm of at least three titanium-containing particles, and even more preferably within at least 6 nm of at least three titanium-containing particles.

The gold particle has an average diameter of less than 15 nm, preferably less than 12 nm, more preferably less than 10 nm, and even more preferably less than 8 nm. The standard deviation of the average diameter of the gold particles is +/−5 nm, preferably +/−2.5 nm. As used herein, the standard deviation is calculated by the following equation:

$$standard deviation = \sqrt{\frac{{\sum }_{i = 1}^{n} \vert x - \overline{x} \vert}{n}}$$

where x is the size of each particle, x̅ is the mean of the n number of particles, and n is at least 500.

The titanium-containing particles may comprise elemental titanium or a titanium oxide, TiOₓ. Preferably, the titanium-containing particles comprise a titanium oxide.

The titanium-containing particles preferably have an average diameter of less than 5 times the average diameter of the gold particles, more preferably an average diameter of less than 4 times the average diameter of the gold particles, even more preferably an average particle diameter of less than 3 times the average diameter of the gold particles, still more preferably an average particle diameter of less than 2 times the average diameter of the gold particles, and yet more preferably an average particle diameter of less than 1.5 times the average diameter of the gold particles.

The amount by weight of the gold particles with respect to the amount of the titanium-containing particles may range from 1:1 to 1:20. Preferably, the weight ratio of gold particles to titanium-containing particles ranges from 1:2 to 1:15, more preferably, from 1:3 to 1:10, and even more preferably, from 1:3 to 1:6.

Preferably, the gold particles are evenly distributed among the titanium-containing particles. As used herein, the term “evenly distributed” means the gold particles are randomly dispersed among the titanium-containing particles with substantially no agglomeration of the gold particles, e.g., less than 10% by weight of the gold particles based on the total weight of the gold particles are in physical contact with another gold particle. Preferably, less than 7.5% by weight of the gold particles based on the total weight of the gold particles are in physical contact with another gold particle, and more preferably, less than 5% by weight of the gold particles based on the total weight of the gold particles are in physical contact with another gold particle.

Preferably, the support is a particle of a refractory oxide capable of withstanding long-term use in an oxidative esterification reactor. Materials that are capable of withstanding prolonged use are able to avoid being crushed or pulverized during use. For example, titanium oxide (TiOₓ) is a support that is highly resistant to acid, but can be mechanically weak when it has a high degree of surface area.

Preferably the support is a particle of γ-, δ-, or θ-alumina, silica, magnesia, zirconia, hafnia, vanadia, niobium oxide, tantalum oxide, ceria, yttria, lanthanum oxide or a combination thereof. Preferably, the support comprises, consists of, or consists essentially of γ-, δ-, or θ-alumina, silica, and magnesia. More preferably, the support comprises, consists of, or consists essentially of silica. As used herein with respect to the support, the phrase “consists essentially of” excludes the presence of materials that would degrade the mechanical strength of the support. Alternatively, “consists essentially of” means that the support comprises at least 95 wt % of the stated material with respect to the total weight of the support.

Preferably, the support has a surface area greater than 10 m²/g, preferably greater than 30 m²/g, preferably greater than 50 m²/g, preferably greater than 100 m²/g, preferably greater than 120 m²/g.

Preferably, the aspect ratio of the catalyst particle is no more than 10:1, preferably no more than 5:1, and preferably no more than 3:1. Although the shape is not limited, preferred shapes for the catalyst particle include spheres, cylinders, rectangular solids, rings, multi-lobed shapes (e.g., cloverleaf cross section), shapes having multiple holes and “wagon wheels;” preferably spheres. Irregular shapes may also be used.

Preferably, at least 90 wt % of the gold particles and titanium-containing particles are in the outer 70% of catalyst volume (i.e., the volume of an average catalyst particle), preferably the outer 60% of catalyst volume, preferably the outer 50%, preferably the outer 40%, preferably the outer 35%, preferably in the outer 30%, preferably in the outer 25%. Preferably, the outer volume of any particle shape is calculated for a volume having a constant distance from its inner surface to its outer surface (the surface of the catalyst particle), measured along a line perpendicular to the outer surface. For example, for a spherical particle the outer x % of volume is a spherical shell whose outer surface is the surface of the particle and whose volume is x % of the volume of the entire sphere. Preferably, at least 95 wt % of the gold particles and titanium-containing particles are in the outer volume of the catalyst, preferably at least 97 wt %, preferably at least 99 wt %. Preferably, at least 90 wt % (preferably at least 95 wt %, preferably at least 97 wt %, preferably at least 99 wt %) of the gold particles and titanium-containing particles are within a distance from the surface that is no more than 30% of the catalyst diameter, preferably no more than 25%, preferably no more than 20%, preferably no more than 15%, preferably no more than 10%, preferably no more than 8%. Distance from the surface is measured along a line which is perpendicular to the surface. Preferably, the gold particles and titanium-containing particles form an eggshell structure on the support particles. The eggshell layer may have a thickness of 500 microns or less, preferably 250 microns or less, and more preferably 100 microns or less.

Preferably, at least 0.1% by weight of the total weight of the gold particles are exposed on a surface of the catalyst. As used herein, the term “exposed” means that at least a portion of the gold particle is not covered by another gold particle or titanium-containing particle, i.e., the reactants can directly contact the gold particle. The gold particles may therefore be disposed within a pore of the support material and still be exposed by virtue of the reactant being able to directly contact the gold particle within the pore. More preferably, at least 0.25% by weight of the total weight of the gold particles are exposed on the surface of the catalyst, even more preferably, at least 0.5% by weight of the total weight of the gold particles are exposed on the surface of the catalyst, and still more preferably, at least 1% by weight of the total weight of the gold particles are exposed on the surface of the catalyst.

Preferably, the average diameter of the catalyst particle is at least 60 microns, preferably at least 100 microns, preferably at least 200 microns, preferably at least 300 microns, preferably at least 400 microns, preferably at least 500 microns, preferably at least 600 microns, preferably at least 700 microns, preferably at least 800 microns; preferably no more than 30 mm, preferably no more than 20 mm, preferably no more than 10 mm, preferably no more than 5 mm, preferably no more than 4 mm. The average diameter of the support and the average diameter of the final catalyst particle are not significantly different.

Preferably, the amount of gold as a percentage of the gold and the support is from 0.2 to 5 wt %, preferably at least 0.5 wt %, preferably at least 0.8 wt %, preferably at least 1 wt %, preferably at least 1.2 wt %; preferably no more than 4 wt %, preferably no more than 3 wt %, preferably no more than 2.5 wt %.

Preferably, the catalyst is produced by precipitating the gold and titanium from an aqueous solution of metal salts in the presence of the support. In one preferred embodiment, the catalyst is produced by an incipient wetness technique in which an aqueous solution of a suitable gold precursor salt and titanium salt is added to a porous inorganic oxide such that the pores are filled with the solution and the water is then removed by drying. The resulting material is then converted into a finished catalyst by calcination, reduction, or other pre-treatments known to those skilled in the art to decompose the gold salts and titanium salts into metals or metal oxides. Preferably, a C₂-C₁₈ thiol comprising at least one hydroxyl or carboxylic acid substituent is present in the solution. Preferably, the C₂-C₁₈ thiol comprising at least one hydroxyl or carboxylic acid substituent has from 2 to 12 carbon atoms, preferably 2 to 8, preferably 3 to 6. Preferably, the thiol compound comprises no more than 4 total hydroxyl and carboxylic acid groups, preferably no more than 3, preferably no more than 2. Preferably, the thiol compound has no more than 2 thiol groups, preferably no more than one. If the thiol compound comprises carboxylic acid substituents, they may be present in the acid form, conjugate base form or a mixture thereof. Especially preferred thiol compounds include thiomalic acid, 3-mercaptopropionic acid, thioglycolic acid, 2-mercaptoethanol and 1-thioglycerol, including their conjugate bases.

In one embodiment of the invention, the catalyst is produced by deposition precipitation in which a porous inorganic oxide is immersed in an aqueous solution containing a suitable gold precursor salt and a titanium salt and is the salts are then made to interact with the surface of the inorganic oxide by adjusting the pH of the solution. The resulting treated solid is then recovered (e.g. by filtration) and then converted into a finished catalyst by calcination, reduction, or other pre-treatments known to those skilled in the art to decompose the gold salts and titanium salts into metals or metal oxides.

Preferably, the process for producing methyl methacrylate (MMA) is performed in an oxidative esterification reactor (OER). The catalyst particles may be present in a slurry or in a catalyst bed, preferably a catalyst bed. The catalyst particles in the catalyst bed typically are held in place by solid walls and by screens or catalyst support grids. In some configurations, the screens or grids are on opposite ends of the catalyst bed and the solid walls are on the side(s), although in some configurations the catalyst bed may be enclosed entirely by screens. Preferred shapes for the catalyst bed include a cylinder, a rectangular solid and a cylindrical shell; preferably a cylinder. The OER further comprises a liquid phase comprising methacrolein, methanol and MMA and a gaseous phase comprising oxygen. The liquid phase may further comprise byproducts, e.g., methacrolein dimethyl acetal (MDA) and methyl isobutyrate (MIB). Preferably, the liquid phase is at a temperature from 40 to 120° C.; preferably at least 50° C., preferably at least 60° C.; preferably no more than 110° C., preferably no more than 100° C. Preferably, the catalyst bed is at a pressure from 0 to 2000 psig (101 kPa to 14 MPa); preferably no more than 2000 kPa, preferably no more than 1500 kPa.

The OER typically produces MMA, along with methacrylic acid and unreacted methanol. Preferably, methanol and methacrolein are fed to the reactor in a methanol:methacrolein molar ratio from 1:10 to 100:1, preferably from 1:2 to 20:1, preferably from 1:1 to 10:1. Preferably, a catalyst bed further comprises inert or acidic materials above and/or below the catalyst. Preferred inert or acidic materials include, e.g., alumina, clay, glass, silica carbide and quartz. Preferably, the inert or acidic material has an average diameter equal to or greater than that of the catalyst, preferably no greater than 20 mm. Preferably, the reaction products are fed to a methanol recovery distillation column which provides an overhead stream rich in methanol and methacrolein; preferably this stream is recycled back to the OER. The bottoms stream from the methanol recovery distillation column comprises MMA, MDA, methacrylic acid, salts and water. In one embodiment of the invention, MDA is hydrolyzed in a medium comprising MMA, MDA, methacrylic acid, salts and water. MDA may be hydrolyzed in the bottoms stream from a methanol recovery distillation column; said stream comprising MMA, MDA, methacrylic acid, salts and water. In another embodiment, MDA is hydrolyzed in an organic phase separated from the methanol recovery bottoms stream. It may be necessary to add water to the organic phase to ensure that there is sufficient water for the MDA hydrolysis; these amounts may be determined easily from the composition of the organic phase. The product of the MDA hydrolysis reactor is phase separated and the organic phase passes through one or more distillation columns to produce MMA product and light and/or heavy byproducts. In another embodiment, hydrolysis could be conducted within the distillation column itself.

One preferred embodiment is a recycle reactor with cooling capacity in the recycle loop. Another preferred embodiment is a series of reactors with cooling and mixing capacity between the reactors.

Preferably, oxygen concentration at a reactor outlet is at least 1 mol %, more preferably at least 2 mol %, even more preferably at least 2.5 mol %, still more preferably at least 3 mol %, yet more preferably at least 3.5 mol %, even yet more preferably at least 4 mol %, and most preferably at least 4.5 mol %, based on the total volume of the gas stream exiting the reactor. Preferably, the oxygen concentration in a gas stream exiting the reactor is no more than 7.5 mol %, preferably no more than 7.25 mol %, preferably no more than 7 mol %, based on the total amount of the gas stream exiting the reactor.

One preferred embodiment of the fixed bed reactor for oxidative esterification is a trickle bed reactor, which contains a fixed bed of catalyst and passes both the gas and liquid feeds through the reactor in the downward direction. In trickle flow, the gas phase is the continuous fluid phase. Thus, the zone at the top of the reactor, above the fixed bed, will be filled with a vapor phase mixture of nitrogen, oxygen, and the volatile liquid components at their respective vapor pressures. Under typical operating temperatures and pressures (50-90° C. and 60-300 psig (400-2000 kPa)), this vapor mixture is inside the flammable envelope if the gas feed is air. Thus, only an ignition source would be required to initiate a deflagration, which could lead to loss of primary containment and harm to the physical infrastructure and personnel in the vicinity. In order to address process safety considerations, a means to operate a trickle bed reactor while avoiding a flammable headspace atmosphere is operation with a gas feed containing a sufficiently low oxygen mole fraction to ensure the oxygen concentration in the vapor headspace is below the limiting oxygen concentration (LOC).

Knowledge of the LOC is required for the fuel mixture, temperature, and pressure of concern. Since the LOC decreases with increasing temperature and pressure, and given that methanol gives a lower LOC than the other two significant fuels (methacrolein and methyl methacrylate), a conservative design chooses a feed oxygen to nitrogen ratio that ensures a composition with less than the LOC at the highest expected operating temperature and pressure. For example, for a reactor operated at up to 100° C. and 275 psig (2 MPa), the feed oxygen concentration in nitrogen should not exceed 7.4 mol %.

### EXAMPLES

### Example

### Catalyst Preparation:

Catalyst was prepared by incipient wetness of on a predominantly spherical pellet modified with titanium, which is present in the final catalyst in its oxide form. 100 g of Fuji Silysia Chemical, Ltd. CARIACT Q-20 silica support material was treated with a titanium salt to add Ti to the support. 4.1 g sodium gold thiosulfate was dissolved in 100 g of water to make an aqueous solution and then placed on the Ti-treated support. The sample was dried at 120° C. for 1 hr followed by calcination at 400° C. for 4 hr. The resulting catalyst comprised 6.5 wt % Ti and 1.4 wt % Au and had slightly higher gold loading near the outer surface of the catalyst.

The gold particle size, as measured by TEM, for the fresh catalyst, the catalyst after a 2000 hour pilot plant trial in a fixed bed bubble column reactor, and after 15 months of additional laboratory aging in a fixed bed bubble column reactor, are shown in Table 1 below. The activity of the fresh catalyst, the catalyst after the 2000 hour pilot plant trial, and the additional laboratory aging exhibited little deactivation of the catalyst.

TABLE 1

| Sample | Average Gold Particle Size (nm) |
|---|---|
| Fresh Catalyst | 3.3 +/− 1.9 standard deviation |
| Catalyst after 2000 Hour Pilot | 5.2 +/− 1.9 standard deviation |
| Plant Trial | |
| Catalyst after 15 Months Additional | 4.6 +/− 1.4 standard deviation |
| Laboratory Aging | |

As seen in Table 1, the catalyst of the invention exhibited excellent longevity and the measured particle size indicated little agglomeration or change in the average size of the gold particles after the 2000 hour pilot plant trial and after 15 months of additional laboratory aging.

1. A catalyst comprising gold particles and titanium-containing particles, wherein the catalyst comprises gold particles within at least 15 nm of at least one titanium-containing particle, and wherein the gold particles have an average diameter of less than 15 nm and a standard deviation of +/−5 nm.

2. The catalyst of claim 1, wherein the gold particles and the titanium-containing particles are disposed on an outer surface of a support material.

3. The catalyst of claim 2, wherein the support material comprises silica.

4. The catalyst of any one of the preceding claims, wherein the titanium-containing particles comprise a titanium oxide.

5. The catalyst of any one of the preceding claims, wherein the gold particles are evenly distributed among the titanium-containing particles.

6. The catalyst of any one of the preceding claims, wherein the gold particles have an average diameter of less than 10 nm and a standard deviation of +/−2.5 nm.

7. The catalyst of any one of the preceding claims, wherein at least 0.1% by weight of the total weight of the gold particles are exposed on a surface of the catalyst.

8. The catalyst of claim 7, wherein at least 0.5% by weight of the total weight of gold particles are exposed on a surface of the catalyst.

9. A method for preparing methyl methacrylate from methacrolein and methanol; said method comprising contacting in a reactor a mixture comprising methacrolein, methanol and oxygen in the presence of a catalyst according to any one of the preceding claims.
